(19) United States
(12) Patent Application Publication
(10) Pub. No.: US 2018/0149611 A1
(43) Pub. Date: May 31, 2018

(54) BIOSENSOR AND METHOD OF FORMING PROBE ON SOLID SURFACE OF BIOSENSOR

(71) Applicants: Caduceus Biotechnology Inc., Hsinchu (TW); HELIOS BIOELECTRONICS INC., Hsinchu County (TW)

(72) Inventors: Golden Tiao, Hsinchu (TW); Rung-Ywan Tsai, Hsinchu (TW); Wen-Yih Chen, Taoyuan City (TW)

(73) Assignees: Caduceus Biotechnology Inc., Hsinchu (TW); HELIOS BIOELECTRONICS INC., Hsinchu County (TW)

(21) Appl. No.: 15/823,610

(22) Filed: Nov. 28, 2017

Related U.S. Application Data
(60) Provisional application No. 62/426,613, filed on Nov. 28, 2016.

(51) Int. Cl. G01N 27/327; G01N 27/406; G01N 27/417

(52) U.S. Cl. CPC G01N 27/3275; G01N 33/54373; G01N 27/417; G01N 27/406

(57) ABSTRACT

A biosensors and a method of forming a probe on a solid surface of a biosensor are disclosed and the method includes the following steps. A unit (a nucleotide or an amino acid) capped with a protecting group is covalently bonded on the solid surface of one of a plurality of sensor cells of the biosensor. At least one cycle of the following steps is performed until a desired number of units is formed: irradiating the one of the plurality of sensor cells, so as to remove the protecting group of the unit; and binding a unit capped with the protecting group to the de-protected unit. The one of the plurality of sensor cells is irradiated to remove the protecting group.

### CROSS REFERENCE TO RELATED APPLICATION

This application claims the priority benefit of U.S.A. provisional application Ser. No. 62/426,613, filed on Nov. 28, 2016. The entirety of the above-mentioned patent application is hereby incorporated by reference herein and made a part of specification.

### BACKGROUND OF THE INVENTION

### Field of the Invention

The invention relates to a biosensor and a method of forming a probe on a solid surface of a biosensor, in particular, to a biosensor and a method of forming a probe on a solid surface of a biosensor in-situ.

### Description of Related Art

Biochips are widely used in the study of genetics, proteomics, pharmaceutical research and clinical detection. Recently, there are many people devoting to improve the quality of the biochips, such as sensitivity, specificity, detection speed, and so on.

The biochip may be formed based on a Field-effect transistor (FET), which has the characteristics of high sensitivity, low requirements of sample amount, and fast screening. In addition, the probes on the surface of the biochip may be formed by DNA synthesis based on phosphoramidite chemistry, peptide synthesis, spotting or printing, and so on. However, for example, in the spotting or printing method, the formed probes have drawbacks such as similar sequence and low variation, and thus is not suitable for customization. In addition, the probes are formed with low density, which fails to achieve high throughput and increases the complexity and the cost of the detection.

### SUMMARY OF THE INVENTION

The invention provides a biosensor in which the probes are covalently bonded to and synthesized in-situ on the solid surface, and the biosensor has good sensitivity and specificity.

The invention provides a method of forming a probe on a solid surface of a biosensor in situ by a photolithography, and the biosensor has good sensitivity and specificity.

The invention provides a biosensor. The biosensor includes a detective platform with a solid surface and an immobilizing probe. The immobilizing probe is covalently bonded to the solid surface and synthesized in-situ on the solid surface by a photolithography process, and the immobilizing probe is able to hybridize or bind to a target object that is to be detected.

The invention provides a method of forming a probe on a solid surface of a biosensor and includes the following steps. A unit capped with a protecting group is covalently bonded on the solid surface of one of a plurality of sensor cells of the biosensor, wherein the unit is a nucleotide or an amino acid. At least one cycle of the following steps is performed until the probe is formed with a desired number of units: irradiating the one of the plurality of sensor cells, so as to remove the protecting group of the unit; and binding a unit capped with the protecting group to the de-protected unit. The one of the plurality of sensor cells is irradiated to remove the protecting group, so as to form the probe.

In an embodiment of the invention, the step of irradiating the one of the plurality of sensor cells includes irradiating the one of the plurality of sensor cells by using a mask to expose the one of the plurality of sensor cells.

In an embodiment of the invention, the step of irradiating the one of the plurality of sensor cells includes irradiating the one of the plurality of sensor cells by using a maskless lithography.

In an embodiment of the invention, the protecting group is a photolabile protecting group or an acid labile protecting group.

In an embodiment of the invention, the photolabile protecting group or acid labile protecting group includes 2-(2-nitrophenyl)propoxycarbonyl (NPPOC) group, α-methyl-2-nitropiperonyloxycarbonyl (MeNPOC) group, thiophenyl-2-(2nitrophenyl)-propoxycarbonyl (SPh-NPPOC) group, 2-(3,4methylenedioxy-6-nitrophenyl)propoxycarbonyl (MNPPOC) group, 6-nitroveratryloxycarbonyl (NVOC) group, Dimethoxybenzoincarbonate (DMBOC) group, 4,4′-Dimethoxytrityl (DMT) group, tert-butyloxycarbonyl (t-Boc) group or the derivative thereof.

In an embodiment of the invention, a light used to irradiate the one of the plurality of sensor cells includes UV light, EUV light, or deep UV light.

In an embodiment of the invention, the probe is covalently bonded onto the one of the plurality of sensor cells with a linker.

In an embodiment of the invention, the probe includes single stranded DNA (ss-DNA), micro RNA (miRNA), aptamer, peptide, protein, or antibody.

In an embodiment of the invention, the ss-DNA, miRNA, or aptamer is synthesized by nucleotides or nucleotide derivatives (such as a Locked Nucleic acid (LNA), or a nucleic acid with a methylated or ethylated phosphate).

In an embodiment of the invention, the peptide, protein, or antibody is synthesized by amino acids or amino acid derivatives.

In an embodiment of the invention, the probe includes fragments of antibody.

In an embodiment of the invention, the probe includes a neutralized DNA (N-DNA) probe of formula (I):

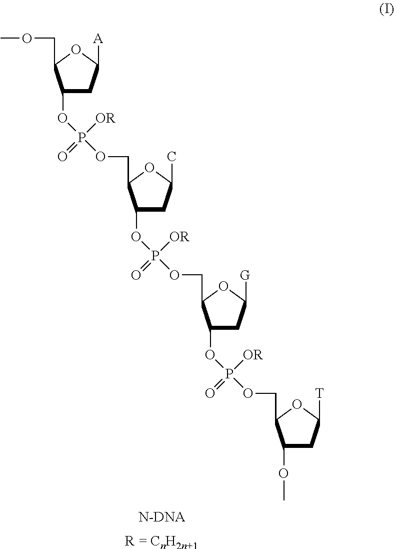

wherein n is 1, 2, or 3 and each A, T, G, and C is any of adenine, thymine, guanine, and cytosine.

In an embodiment of the invention, the probe includes a LNA of formula (II):

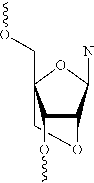

wherein N is adenine, thymine, guanine, or cytosine.

In an embodiment of the invention, the probe is label-free probe.

Based on the above, the invention provides a method of forming a probe on a solid surface of a biosensor in situ by a photolithography. Since the probe is synthesized on the solid surface in situ, the probe may be easily designed according to the needs and the density of the probes can be increased. In addition, the vile of the design of mask patterns is based on the sequence of probes to reduce the number of masks and shorten the photosynthesis time and cost. Accordingly, the sensitivity, the specificity, the rapid reaction, or low cost of the biosensor may be achieved.

In order to make the aforementioned features and advantages of the invention more comprehensible, embodiments are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIGS. 1A-1N illustrate a method of forming a probe on a solid surface of a biosensor according to some embodiments of the invention.

FIGS. 2A-2I illustrate masks according to some embodiments of the invention.

### DESCRIPTION OF THE EMBODIMENTS

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

The present invention provides a biosensor. The biosensor includes a wafer, the wafer includes a plurality of chips arranged in an array, and each chip includes at least one sensor cell (also referred as biochip). Accordingly, the biosensor is a micro- or nano-array biochips, and the micro- or nano-array biochips are made by a traditional semiconductor process such as a process for manufacturing a silicon wafer. The wafer may be a silicon wafer, and has a diameter of 2″, 4″, 6″, 8″, or 12″, for example. In some embodiments, there are N×N sensor cells arranged in an array in one chip, and N is 1 to 1,000,000. Each sensor cell has a transistor with a solid surface and an immobilizing probe in situ synthesized on the solid surface. In some embodiments, the transistor has nanostructure, and is a nanowire FET, MOSFET, an extended MOSFET, or other suitable transistor. Generally, the transistor includes a substrate, source and drain disposed on the substrate, a gate insulating layer between the gate and the source and drain. A gate is disposed on the top of the gate insulating layer. In some embodiments, materials of the source and drain include doped Si or other suitable semiconductor material. A material of the gate insulating layer includes silicon oxide, silicon nitride, silicon oxynitride, polymer, the like, or a combination thereof. A channel layer is disposed on the top of the gate insulating layer of the transistor. The channel layer is a semiconductor layer or a conductive layer, and includes silicon, carbon, carbon nanotube, Ge, graphene, graphene oxide, the like, or a combination thereof. The channel layer can be a micro-pad, a nano-pad, a micro-wire a nano-wire or other suitable structure. A size of the channel layer may range from 10 nm to 200 um (i.e., length, L)×1 nm to 10 um (i.e., width, W) or 10 nm to 200 um (i.e., length, L)×10 nm to 200 um (i.e., width, W). There may be a liquid serving as a gate and covering the probe in situ synthesized in a micro- or nano-structure such as a well, and there also may be a reference electrode disposed in the liquid.

The immobilizing probe is covalently bonded to the surface of the channel layer and synthesized in-situ on the channel layer by a photolithography process, and the immobilizing probe is able to hybridize or bind to a target object that is to be detected. In some embodiments, an area of the probe in the sensor cell ranges from 10 nm to 200 um×1 nm to 10 um, for example. The immobilizing probe includes ss-DNA, miRNA, aptamer, peptide, protein, antibody, fragments of antibody, or other suitable probe. Based on the sequence, the ss-DNA, miRNA, or aptamer is chemically synthesized using nucleotides (i.e., adenine, thymine, guanine, or cytosine) or nucleotide derivatives (such as a LNA, or a nucleic acid with a methylated or ethylated phosphate) by photolithography, and the peptide, protein, or antibody is chemically synthesized using amino acids (i.e., Ala, Arg, Asn, Asp, Cys, Glu, Gln, Gly, His, Ile, Leu, Lys, Met, Phe, Pro, Ser, Thr, Trp, Tyn, or Val) by photolithography.

In some embodiments, the immobilizing probe is a N-DNA probe of formula (I):

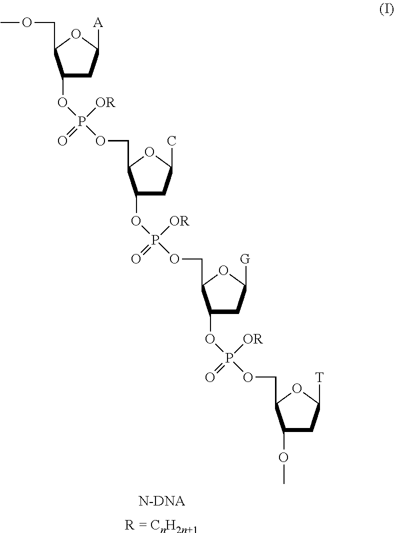

wherein n is 1, 2, or 3 and each A, T, G, and C is any of adenine, thymine, guanine, and cytosine. In some embodiments, the probe can hybridize to a complementary target nucleic acid that is to be detected. In some embodiments, the N-DNA modifies the charged oxygen ions (O⁻) on the phosphate backbone of DNA via methylation, ethylation, propylation, or alkylation, so that the backbone is not charged or partially uncharged after this modification to increase the hybridization efficiency, sensitivity and to make the signal clear.

In some embodiments, the immobilizing probe includes a LNA of formula (II):

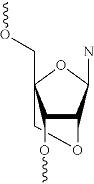

wherein N is adenine, thymine, guanine, or cytosine.

In some embodiments, the immobilizing probe is a label-free probe. In some embodiments, the immobilizing probe has a length ranging from 5 to 500 units, and the units are nucleotides or amino acids. In other words, the immobilizing probe has 5 to 500 mers of nucleotides or 5 to 500 residues of amino acids. The target object is a molecular in a sample, such as ss-DNA, miRNA, aptamer, peptide, protein, antibody, antigen, or other suitable object. In some embodiments, when the target object is hybridized or bound to the immobilizing probe, a surface charge of the solid surface is changed, and thus an electrical signal is obtained and measured. Accordingly, the target object is detected.

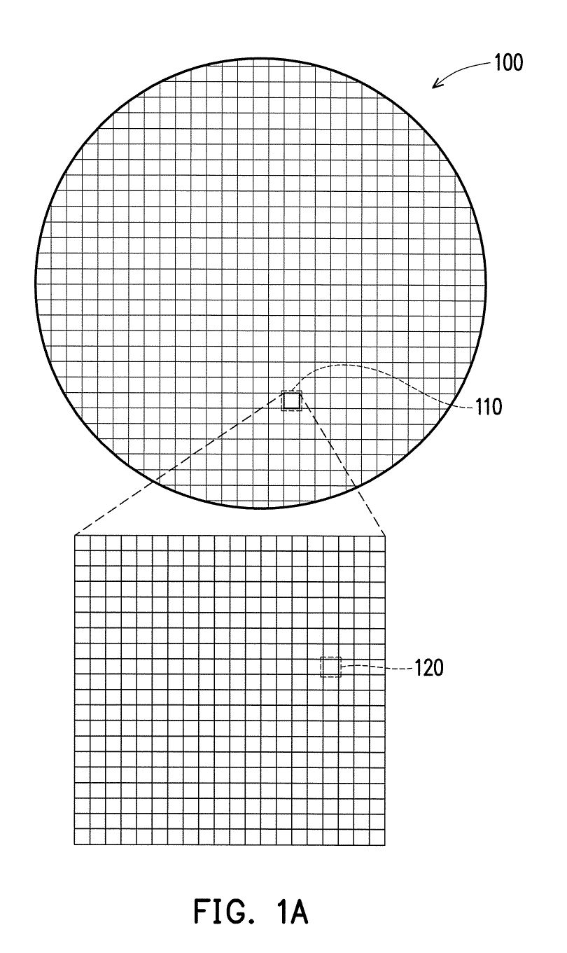

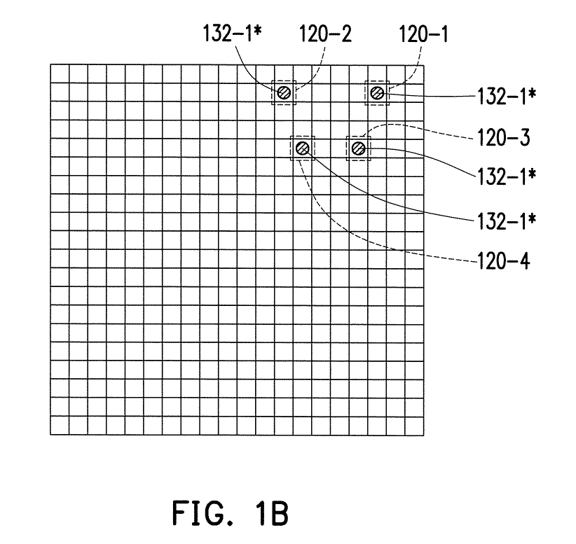

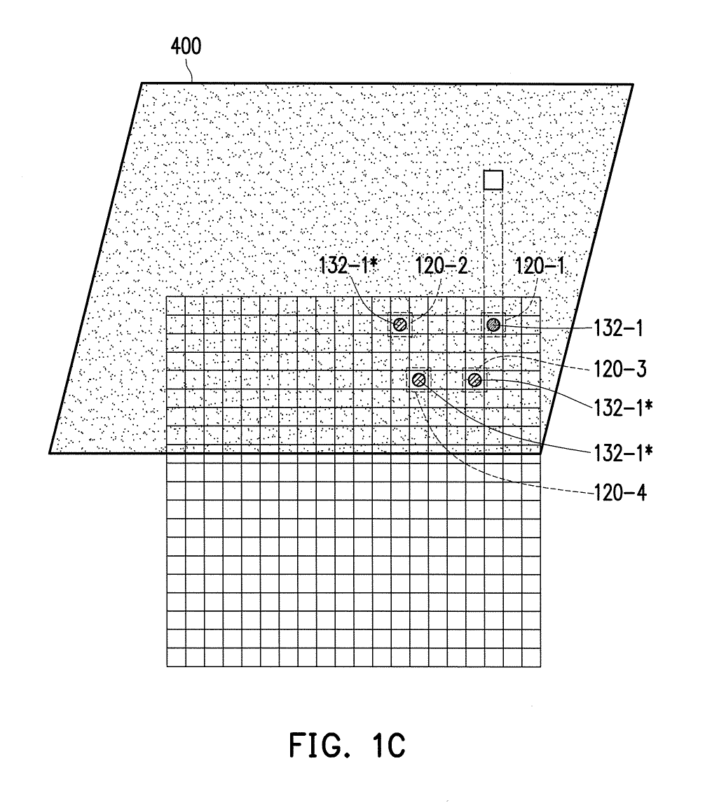

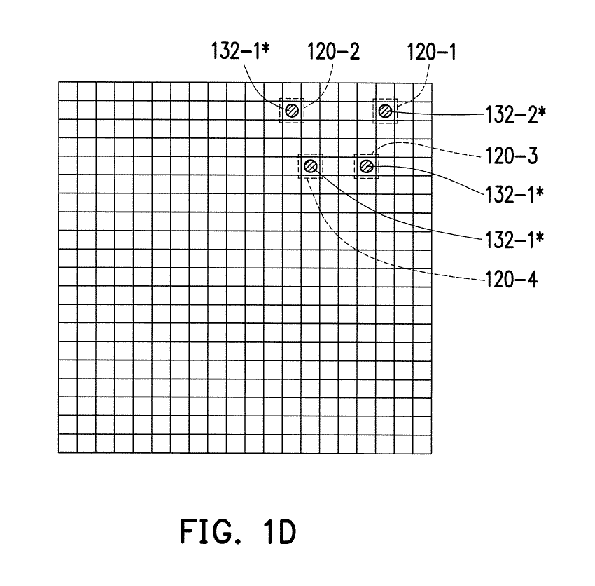

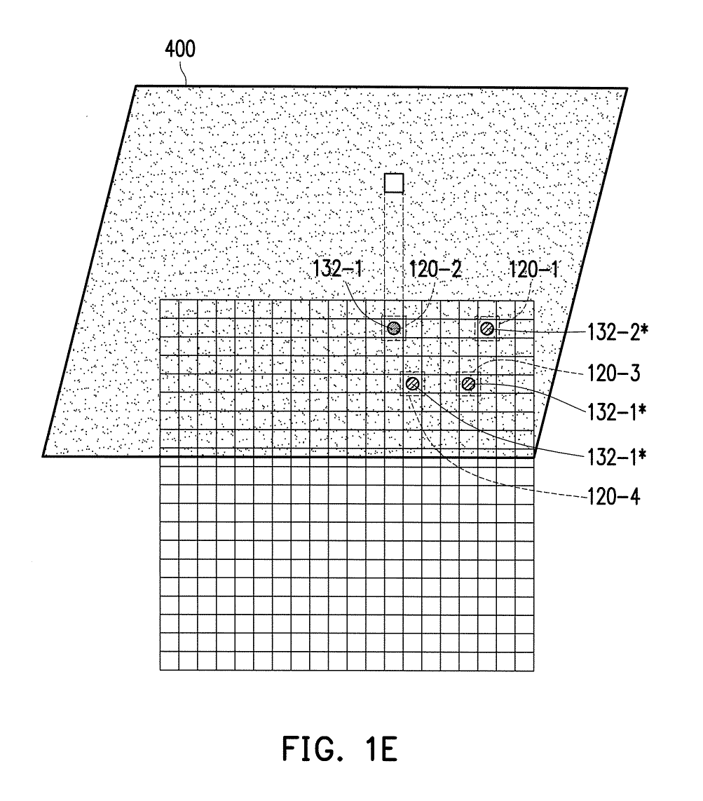

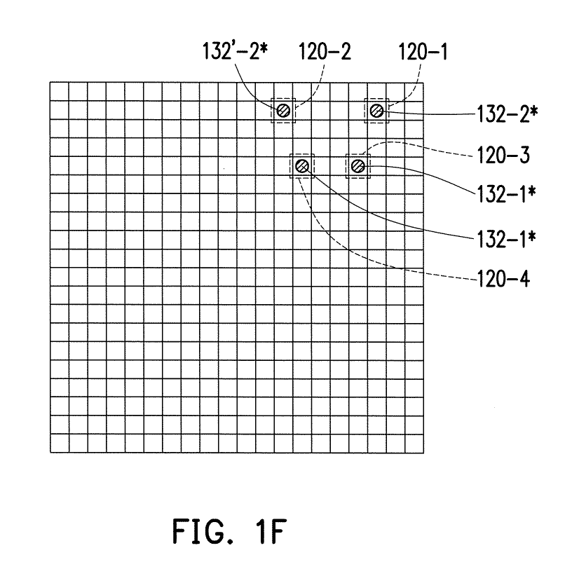

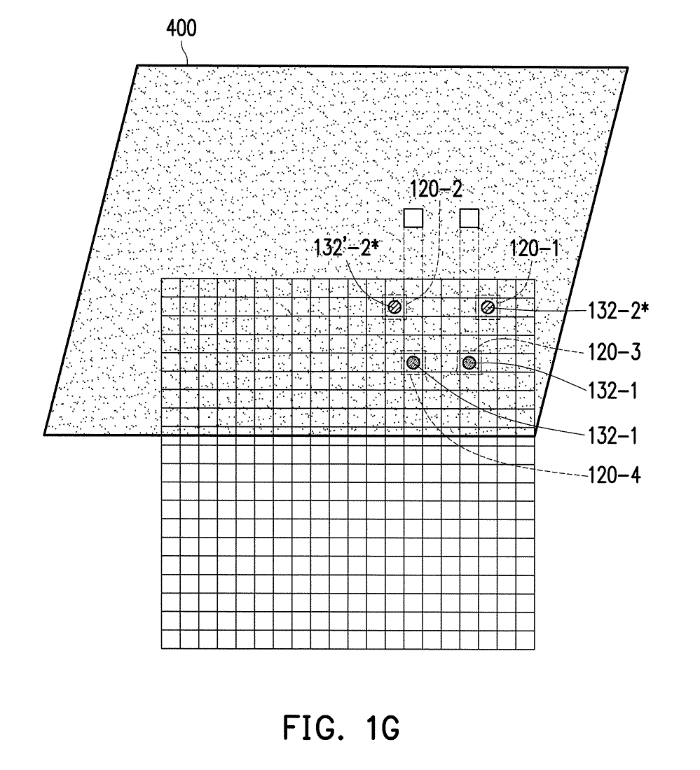

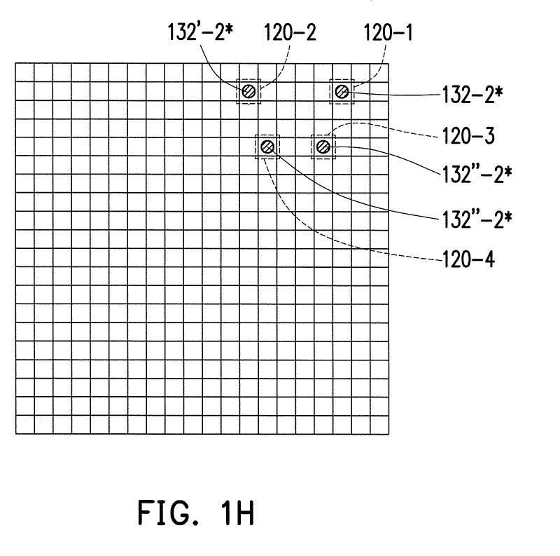

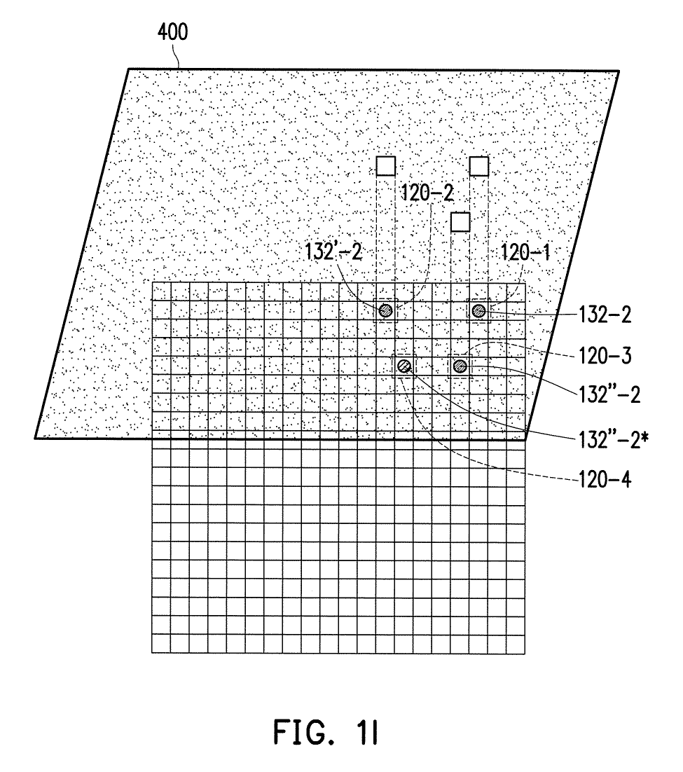

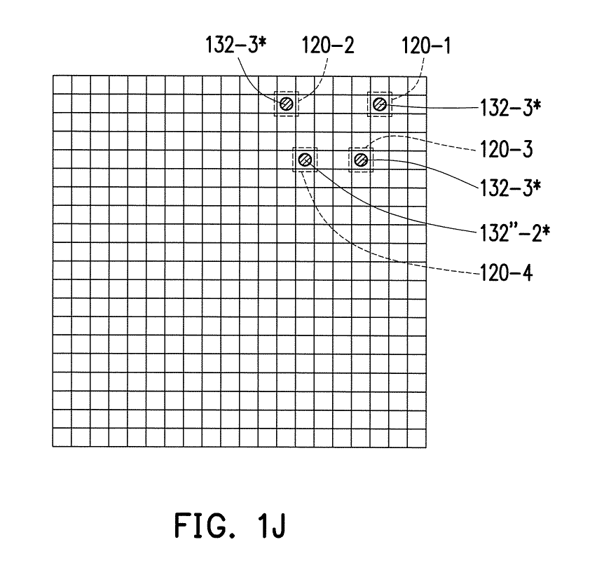

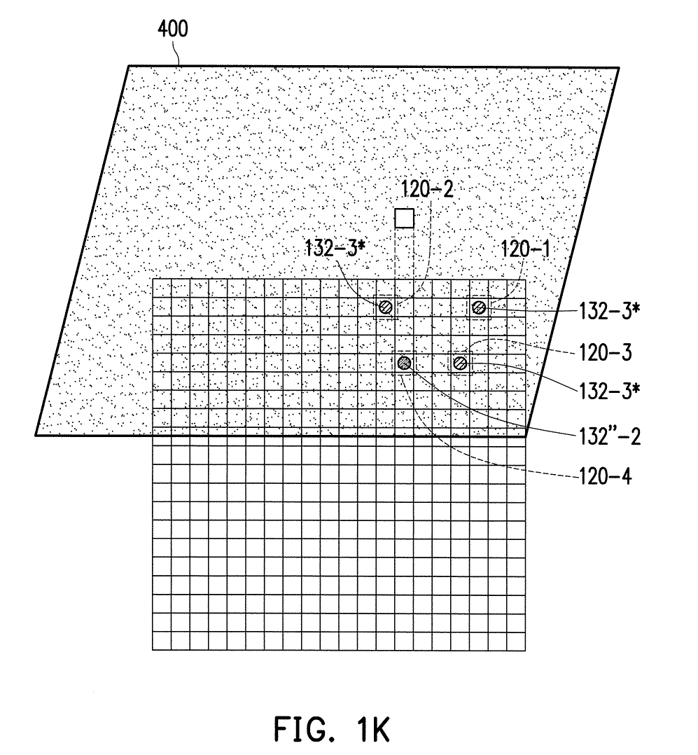

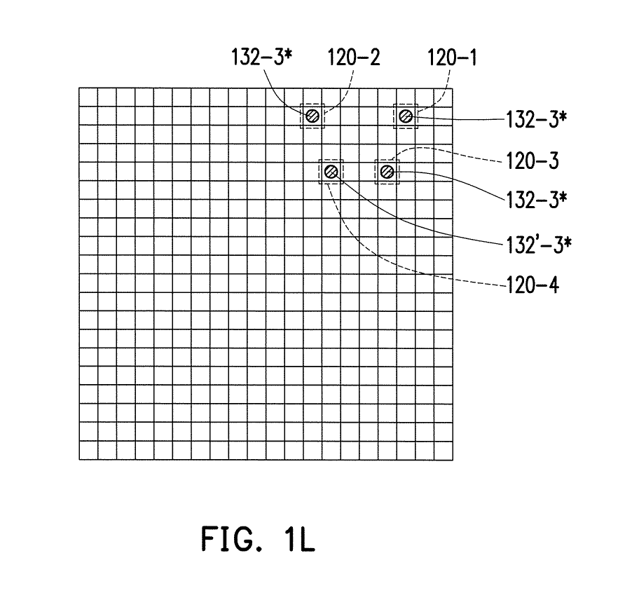

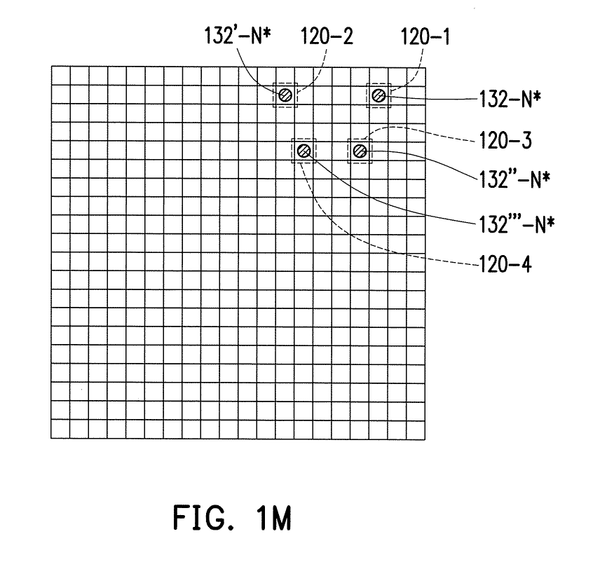

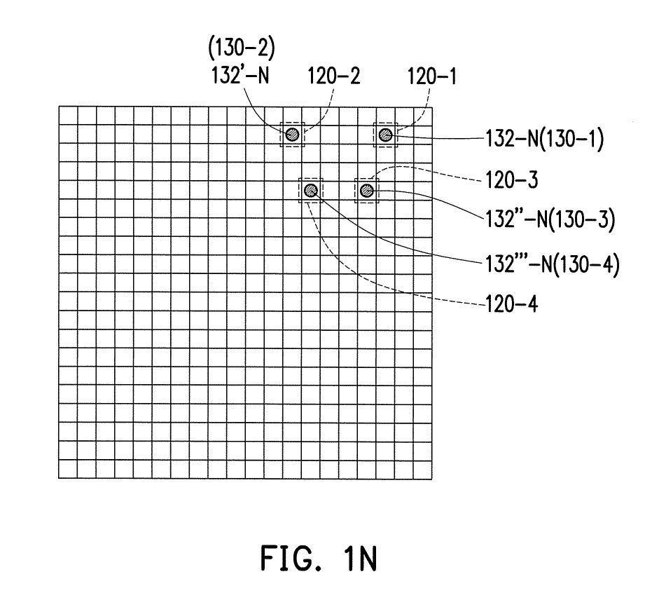

The present invention further provides a method of forming a probe on a solid surface of a biosensor. In some embodiments, a ssDNA probe is used as the immobilizing probe, the FET is used as the transistor of the biosensor, and the present invention is not limited by this platform. FIGS. 1A-1N illustrate a method of forming a probe on a solid surface of a biosensor according to some embodiments of the invention.

Referring to FIG. 1A, a wafer 100 is provided. The wafer 100 includes a plurality of chips 110, and each chip 110 includes M×M sensor cells 120 arranged in an array. Then, a plurality of transistors (not shown) are formed on the sensor cells 120 respectively, and tops of the transistors provide solid surfaces. In some embodiments, the wafer 100 is a silicon wafer, and the transistors are FETs and fabricated by a traditional semiconductor process, for example.

Then, the probes are in-situ formed on the solid surfaces of the sensor cells respectively. It is noted that for clarity, the probes formed on four sensor cells 120-1, 120-2, 120-3, 120-4 are illustrated, but the present invention is not limited thereto. In addition, only one probe is formed on one sensor cell, but a plurality of probes may be formed on one sensor cell.

Referring to FIG. 1B, a first unit 132-1* capped with a protecting group (i.e., a first protected unit) is covalently bonded on at least one of the sensor cells 120-1, 120-2, 120-3, 120-4, wherein the first unit 132-1* is a nucleotide. In some embodiments, the first unit 132-1* is coated on the solid surface and attached thereto via a linker, and the first unit 132-1* is also referred to the first unit of the probe on the sensor cells 120-1, 120-2, 120-3, 120-4. The protecting group is a photolabile protecting group, an acid labile protecting group or other suitable protecting group which is able to be removed by photolithography. In some embodiments, as the probe is ss-DNA, DNA aptamer or the like, the first unit 132-1* is a deoxynucleotide (dNTP) selected from dATP, dCTP, dGTP and dTTP, and the protecting group is a 2-(2-nitrophenyl)propoxycarbonyl (NPPOC) group, α-methyl-2-nitropiperonyloxycarbonyl (MeNPOC) group, thiophenyl-2-(2nitrophenyl)-propoxycarbonyl (SPh-NPPOC) group, 2-(3,4methylenedioxy-6-nitrophenyl)propoxycarbonyl (MNPPOC) group, 6-nitroveratryloxycarbonyl (NVOC) group, Dimethoxybenzoincarbonate (DMBOC) group, 4,4′-Dimethoxytrityl (DMT) group, or their derivatives thereof such as or the like. In alternative embodiments, as the probe is miRNA, RNA aptamer or the like, the first unit 132-1* is a nucleotide (NTP) selected from ATP, CTP, GTP and UTP, and the protecting group is a NPPOC group, MeNPOC group, SPh-NPPOC group, MNPPOC group, NVOC group, DMBOC group, or DMT group and their derivative thereof such as or the like. In some embodiments, the first units 132-1* of the probes on the sensor cells 120-1, 120-2, 120-3, 120-4 are of the same type and thus are formed simultaneously in the same processes, but the present invention is not limited thereto. In other words, in alternative embodiments, at least two of the first units of the probes on the sensor cells may be different, and thus are formed separately in different processes.

After all of the first units of the probe are formed, processes of forming second units of the probes are performed. Depending on a desired number (i.e., N ranging from 5 to 500) of units in the probe on the sensor cell, a cycle for forming a unit is repeated several times (i.e., N−1), which includes the photo-exposure process, the nucleotide coupling process, the oxidation process and the drying process.

Referring to FIG. 1C, at least one of the sensor cells 120-1, 120-2, 120-3, 120-4 is irradiated, so that the protecting group is removed from the first unit 132-1* to form a first de-protected unit 132-1. In some embodiments, a mask 400 is provided to expose the sensor cell 120-1 and shield other sensor cells such as sensor cells 120-2, 120-3, 120-4, and then a light source is provided to irradiate the wafer 100. Since the sensor cell 120-1 is exposed by the mask, the protecting group of the first unit 132-1* is irradiated and removed to form the first de-protected unit 132-1. In the photo-exposure process (also referred as a photodeprotection process or an array patterning process), the irradiation may be a UV light exposure with a wavelength of 365 nm, an intensity ranging from 7 to 80 mW/cm², and an irradiation time of 10 to 60 seconds, for example. In an embodiment, a light used to perform irradiation may be EUV light, deep UV light or other suitable light. Thereafter, the mask 400 is removed. In alternative embodiments, the photo-exposure process may be performed by using a maskless lithography.

Referring to FIG. 1D, a second unit 132-2* (i.e., a second protected unit of the probe) capped with the protecting group is bound to the first de-protected unit 132-1 on the sensor cell 120-1. In some embodiments, the second unit 132-2* may be the same or different from the first unit 132-1*. Since the first units 132-1* on other sensor cells 120-2, 120-3, 120-4 are protected by the protecting groups, the second unit 132-2* is merely bound to the first de-protected unit 132-1 on the sensor cell 120-1. In some embodiments, after binding, an oxidation reaction and a drying process are performed. The oxidation reaction is iodine oxidation such as performing by using water and pyridine for 1 to 10 seconds, for example. The drying process is performed by using a He gas for 10 to 30 seconds, for example.

Referring to FIG. 1E, after the probe on the sensor cell 120-1 is partially synthesized by the first and second units 132-1, 132-2*, at least one of the sensor cells 120-2, 120-3, 120-4 is irradiated, so that the protecting group is removed from the first unit 132-1* to form a first de-protected unit 132-1. In some embodiments, a mask 400 is provided to expose the sensor cell 120-2 and shield other sensor cells such as sensor cells 120-1, 120-3, 120-4, and then a light source is provided to irradiate the wafer 100. Since the sensor cell 120-2 is exposed by the mask, the protecting group of the first unit 132-1* is irradiated and removed. Thereafter, the mask 400 is removed.

Referring to FIG. 1F, a second unit 132′-2* (i.e., a second protected unit of the probe) capped with the protecting group is bound to the first de-protected unit 132-1 on the sensor cell 120-2. In some embodiments, the second unit 132′-2* is different from the second unit 132-2*. Since the second unit 132-2* on the sensor cell 120-1 and the first units 132-1* on other sensor cells 120-3, 120-4 are protected by the protecting groups, the second unit 132′-2* is merely bound to the first de-protected unit 132-1 on the sensor cell 120-2. In some embodiments, after binding, an oxidation reaction and a drying process are performed.

Referring to FIG. 1G, after the probes on the sensor cells 120-1, 120-2 are partially synthesized by the first and second units, at least one of the sensor cells 120-3, 120-4 is irradiated, so that the protecting group is removed from the first unit 132-1* to form a first de-protected unit 132-1. In some embodiments, since the second units of the probes on the sensor cells 120-3, 120-4 are of the same kind, the second units can be formed simultaneously by the same photo-exposure and nucleotide coupling processes. In other words, the second units 132″-2* of the probe on the sensor cells 120-3, 120-4 are different from the second units 132-2*, 132′-2* on the sensor cells 120-1, 120-2, and thus they are not formed in the same process. Particularly, a mask 400 is provided to expose the sensor cells 120-3, 120-4 and shield other sensor cells such as sensor cells 120-1, 120-2, and then a light source is provided to irradiate the wafer 100. Since the sensor cells 120-3, 120-4 is exposed by the mask, the protecting group of the first unit 132-1* is irradiated and removed. Thereafter, the mask 400 is removed.

Referring to FIG. 1H, second units 132″-2* (i.e., a second protected unit of the probe) each capped with the protecting group are bound to the first de-protected units 132-1 respectively on the sensor cells 120-3, 120-4. In some embodiments, the second units 132″-2* of the probe on the sensor cells 120-3, 120-4 are different from the second units 132-2*, 132′-2* on the sensor cells 120-1, 120-2. Since the second unit 132-2*, 132′-2* on other sensor cells 120-1, 120-2 are protected by the protecting groups, the second units 132″-2* are merely bound to the first de-protected units 132-1 on the sensor cell 120-3, 120-4. In some embodiments, after binding, an oxidation reaction and a drying process are performed.

After all of the second units of the probe are formed, processes of forming third units of the probes are performed. Referring to FIG. 1I, at least one of the sensor cells 120-1, 120-2, 120-3 is irradiated, so that the protecting group is removed from the second unit 132-2*, 132′-2*, 132″-2* to form the second de-protected unit 132-2, 132′-2, 132″-2. In some embodiments, a mask 400 is provided to expose the sensor cells 120-1, 120-2, 120-3 and shield other sensor cell such as sensor cell 120-4, and then a light source is provided to irradiate the wafer 100. Since the sensor cells 120-1, 120-2, 120-3 are exposed by the mask, the protecting groups of the second unit 132-2*, 132′-2*, 132″-2* are irradiated and removed. Thereafter, the mask 400 is removed.

Referring to FIG. 1J, third units 132-3* (i.e., third protected units of the probes) capped with the protecting group are bound to the second de-protected units 132-2, 132′-2, 132″-2 respectively on the sensor cells 120-1, 120-2, 120-3. Since the second unit 132″-2* on the sensor cell 120-4 is protected by the protecting groups, the third units 132-3* are merely bound to the second de-protected units 132-2, 132′-2, 132″-2 on the sensor cell 120-1, 120-2, 120-3. In some embodiments, after binding, an oxidation reaction and a drying process are performed.

Referring to FIG. 1K, the sensor cell 120-4 is irradiated, so that the protecting group is removed from the second unit 132″-2* to fonn the second de-protected unit 132″-2. Referring to FIG. 1L, a third unit 132′-3* (i.e., a third protected unit of the probe) capped with the protecting group is bound to the second de-protected unit 132″-2 on the sensor cell 120-4.

Referring to FIG. 1M, after the Nth protected units 132-N*, 132′-N*, 132″-N*, 132′″-N* are bound to the (N−1)th units 132-(N−1) (not shown). In some embodiments, the Nth protected units 132-N*, 132′-N*, 132″-N*, 132′″-N* are different, for example. In alternative embodiments, the Nth protected units of the probes may be of the same type or partially different. Referring to FIG. 1N, a final photo-exposure is performed on the Nth units 132-N*, 132′-N*, 132”-N*, 132′″-N* to form Nth de-protected units 132-N, 132′-N, 132″-N, 132′″-N, so that the probes 130-1, 130-2, 130-3, 130-4 are formed. The probe 130-1 is in-situ formed on the sensor cell, and the probe 130-1 includes N units and is synthesized by the first unit 131-1, the second unit 131-2, the third unit 131-3, . . . , the (N−1)th unit 132-(N−1), and the Nth unit 132-N. In some embodiments, the number of the units of the probes 130-1, 130-2, 130-3, 130-4 may be the same or different.

In alternative embodiments, as the probe is peptide, protein, aptamer, antibody or the like, the unit is an amino acid selected from Ala, Arg, Asn, Asp, Cys, Glu, Gln, Gly, His, Ile, Leu, Lys, Met, Phe, Pro, Ser, Thr, Trp, Tyn, and Val, and the protecting group is a tert-butyloxycarbonyl (t-Boc) group, the derivative thereof such as or the like. In this embodiment, depending on a desired number (i.e., N) of amino acid residues in the probe, a cycle of a photo-exposure process (also referred as a photodeprotection process), a neutralization process for forming —NH₂, an amino acid coupling process, and a drying process is repeated several times (i.e., N−1). The photo-exposure process may be performed by using a mask lithography or a maskless lithography. In the photo-exposure process, the irradiation may be a UV light exposure with a wavelength of 365 nm, an intensity ranging from 7 to 80 mW/cm², and a irradiation time of 10 to 60 seconds, for example. The amino acid coupling process is performed for 10 to 60 seconds, and the drying process is performed by using a He gas for 10 to 30 seconds, for example.

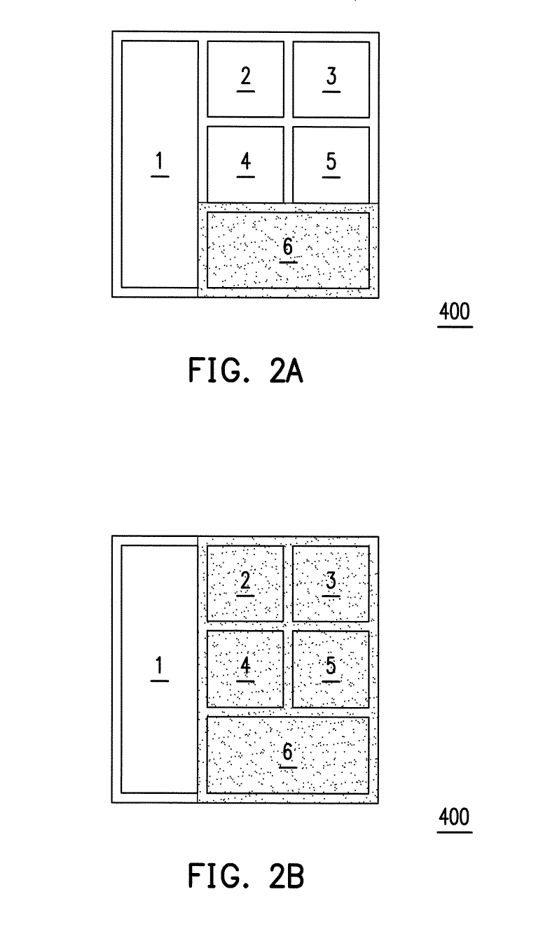

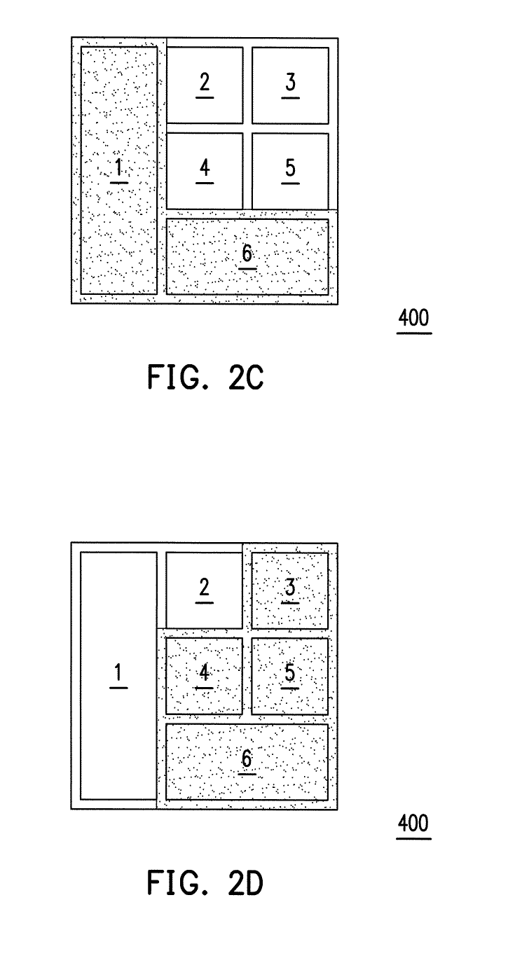

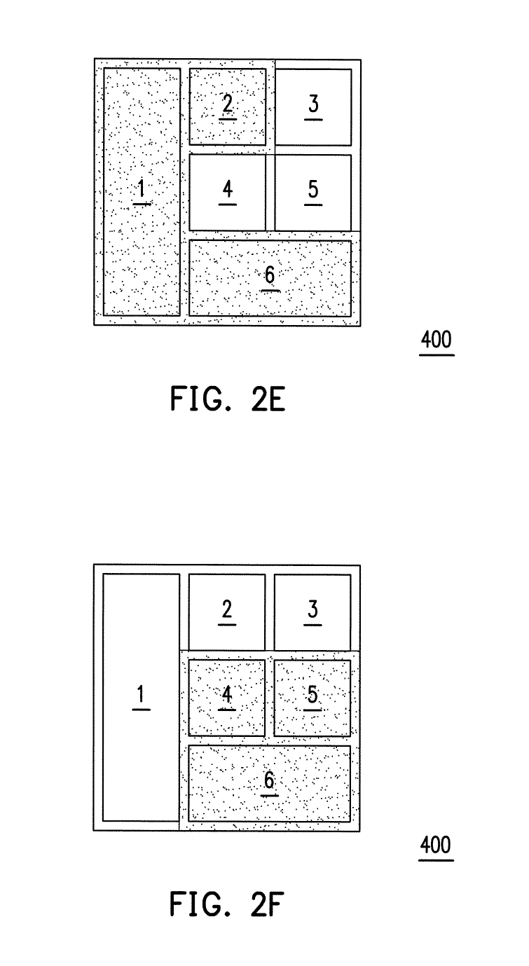

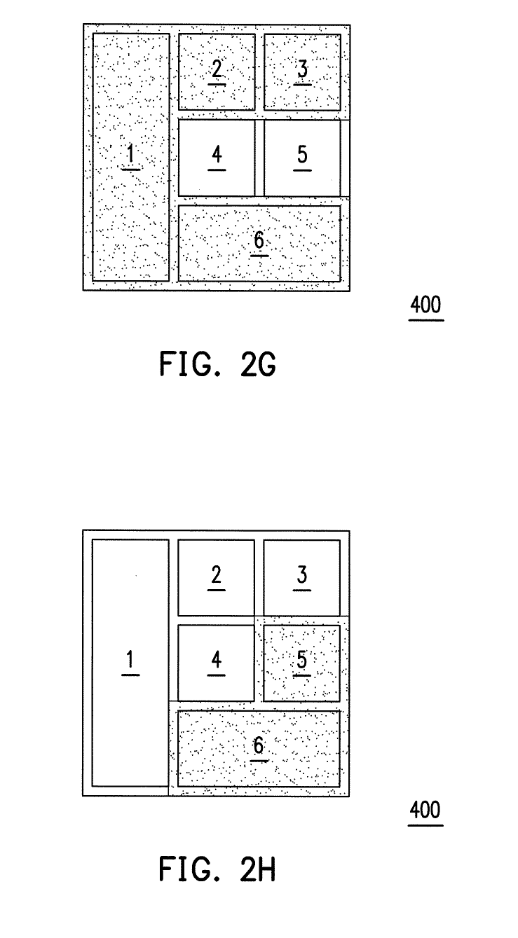

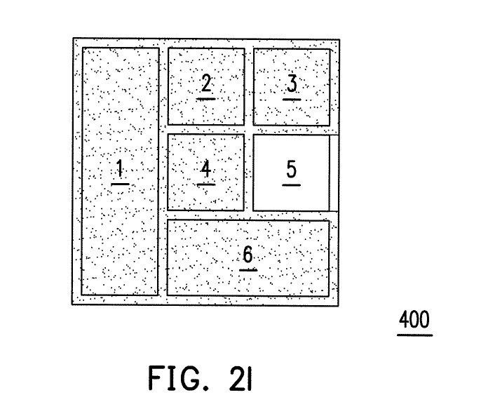

FIGS. 2A-2I illustrate masks according to some embodiments of the invention. In some embodiments, as shown in FIGS. 2A to 2I, a plurality of masks 400 is provided, and each mask 400 shields at least one chip 1-6. By sequentially using the masks 400 with of FIGS. 2A to 2I, the probe is formed onto the chip 1-6 with a desired sequence. In some embodiments, these probes may be used as control, so as to assure the specificity and accuracy of the biochip.

To sum up, the invention provides a method of forming a probe on a solid surface of a biosensor in situ by a photolithography. Since the probe is synthesized on the solid surface in situ, the probe may be easily designed according to the needs and the density of the probes can be increased. Furthermore, the units of the same type of the probes on different sensor cells may be formed simultaneously, and thus the cost and the complexity of forming the probes can be reduced. In addition, by combining the semiconductor techniques and the in-situ synthesis of the probes, the sensitivity, the specificity, the rapid reaction, or low cost of the biosensor may be achieved.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A biosensor comprising:
a detective platform, with a solid surface; and
an immobilizing probe, wherein the immobilizing probe is covalently bonded to the solid surface and synthesized in-situ on the solid surface by a photolithography process, and the immobilizing probe is able to hybridize or bind to a target object that is to be detected.

2. A method of forming a probe on a solid surface of a biosensor comprising:
covalently bonding a unit capped with a protecting group on the solid surface of one of a plurality of sensor cells of the biosensor, wherein the unit is a nucleotide or an amino acid;
performing at least one cycle of the following steps until the probe is formed with a desired number of units:
irradiating the one of the plurality of sensor cells, so as to remove the protecting group of the unit; and
binding a unit capped with the protecting group to the de-protected unit; and
irradiating the one of the plurality of sensor cells to remove the protecting group, so as to form the probe.

3. The method as claimed in claim 2, wherein the step of irradiating the one of the plurality of sensor cells comprises irradiating the one of the plurality of sensor cells by using a mask to expose the one of the plurality of sensor cells.

4. The method as claimed in claim 2, wherein the step of irradiating the one of the plurality of sensor cells comprises irradiating the one of the plurality of sensor cells by using a maskless lithography.

5. The method as claimed in claim 2, wherein the protecting group is a photolabile protecting group or an acid labile protecting group.

6. The method as claimed in claim 5, wherein the photolabile protecting group or acid labile protecting group comprises 2-(2-nitrophenyl)propoxycarbonyl (NPPOC) group, α-methyl-2-nitropiperonyloxycarbonyl (MeNPOC) group, thiophenyl-2-(2nitrophenyl)-propoxycarbonyl (SPh-NPPOC) group, 2-(3,4methylenedioxy-6-nitrophenyl)propoxycarbonyl (MNPPOC) group, 6-nitroveratryloxycarbonyl (NVOC) group, Dimethoxybenzoincarbonate (DMBOC) group, 4,4′-Dimethoxytrityl (DMT) group, tert-butyloxycarbonyl (t-Boc) group or their derivative thereof.

7. The method as claimed in claim 2, wherein a light used to irradiate the one of the plurality of sensor cells comprises UV light, EUV light, or deep UV light.

8. The method as claimed in claim 2, wherein the probe is covalently bonded onto the one of the plurality of sensor cells with a linker.

9. The method as claimed in claim 2, wherein the probe comprises ss-DNA, miRNA, aptamer, peptide, protein, or antibody.

10. The method as claimed in claim 9, wherein the ss-DNA, miRNA, or aptamer is synthesized by nucleotides or nucleotide derivatives.

11. The method as claimed in claim 9, wherein the peptide, protein, or antibody is synthesized by amino acids or amino acid derivatives.

12. The method as claimed in claim 2, wherein the probe comprises fragments of antibody.

13. The method as claimed in claim 2, wherein the probe comprises a neutralized DNA (N-DNA) probe of formula (I): 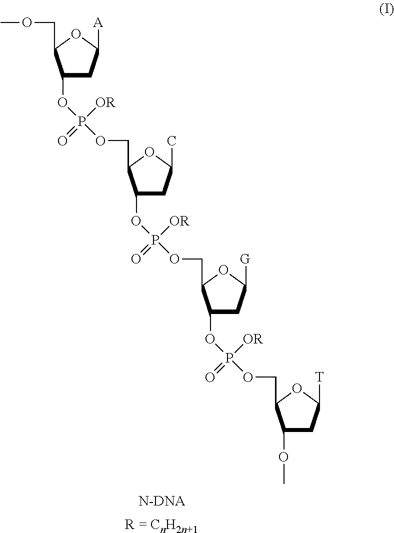
wherein n is 1, 2, or 3 and each A, T, G, and C is any of adenine, thymine, guanine, and cytosine.

14. The method as claimed in claim 2, wherein the probe comprises a Locked Nucleic Acid (LNA) of formula (II): 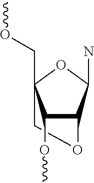
wherein N is adenine, thymine, guanine, or cytosine.

15. The method as claimed in claim 2, wherein the probe comprises a nucleic acid with a methylated or ethylated phosphate.

16. The method as claimed in claim 2, wherein the probe is label-free probe.
